(19) United States
(12) Patent Application Publication
(10) Pub. No.: US 2025/0186451 A1
(43) Pub. Date: Jun. 12, 2025

(54) METHODS OF TREATING CANCER WITH IAP ANTAGONIST COMPOUNDS AND COMBINATION THERAPIES

(71) Applicants: Otsuka Pharmaceutical Co., Ltd., Tokyo (JP); Board of Regents, The University of Texas System, Austin, TX (US)

(72) Inventors: Martin John SIMS, Warsesley (GB); George Albert WARD, Bury St. Edmunds (GB); Tomoko SMYTH, Coton (GB); Gautam BORTHAKUR, Houston, TX (US)

(21) Appl. No.: 18/843,651

(22) PCT Filed: Mar. 3, 2023
(86) PCT No.: PCT/US2023/063669

§ 371 Date: Sep. 3, 2024

Related U.S. Application Data
(60) Provisional application No. 63/316,680, filed on Mar. 4, 2022.

(51) Int. Cl. A61K 31/5377; A61K 31/573; A61K 31/635; A61P 35/02

(52) U.S. Cl. CPC A61K 31/5377; A61K 31/573; A61K 31/635; A61P 35/02

(57) ABSTRACT

The present disclosure relates generally to use of a compound of formula (I), also named ASTX660 in combination therapies for treating cancer, in particular leukemia.

### CROSS REFERENCE TO RELATED APPLICATIONS

This application claims the benefit under 35 U.S.C. § 119 (e) of U.S. Provisional Application No. 63/316,680 filed Mar. 4, 2022, which is hereby incorporated by reference herein in its entirety.

### FIELD

The present application relates to methods of treating cancer using IAP antagonist compounds and combination therapies comprising IAP antagonist compounds.

### BACKGROUND

Inhibitors of apoptosis proteins (LAPs) are a family of antiapoptotic proteins that block cell death (apoptosis) and promote cell cycle progression. Cancer cells over-express IAPs resulting in cancer cell survival and tumor growth. IAP overexpression is a prognostic marker in a variety of solid tumors and hematologic malignancies. Eight distinct human IAPs have been characterized: XIAP, hILP-2, c-IAP1, c-IAP2, ML-IAP, NAIP, Survivin and Apollon. As IAPs are preferentially expressed in malignant cells, suppressing the IAPs can potentially reestablish apoptotic pathways and induce cancer cell death.

There is a need for cancer therapies that can overcome certain drawbacks such as the development of resistance to anti-cancer drugs.

### SUMMARY

As resistance to anti-cancer agents becomes more prevalent, targeting IAPs represents a strategy to resensitize refractory cancer cells to existing chemotherapies. In some embodiments, provided herein is a method for treating cancer in a subject comprising administering to the subject in need thereof a compound of formula I:

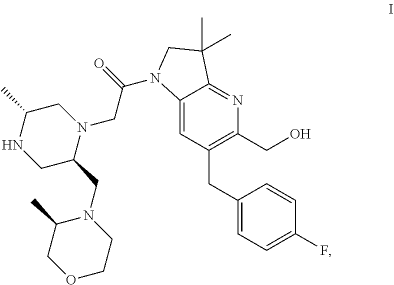 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and
a B cell leukemia/lymphoma-2 (Bcl-2) inhibitor, or a pharmaceutically acceptable salt thereof.

In some embodiments, provided herein is a method for treating cancer in a subject comprising administering to the subject in need thereof a compound of formula I, or a pharmaceutically acceptable salt thereof; and a compound of formula II:

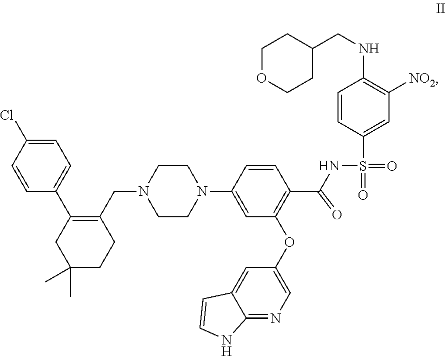 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt thereof.

In some embodiments, provided herein is a method for sensitizing a cancer to a chemotherapy when the cancer is refractory to said chemotherapy, the method comprising administering to a subject in need thereof a compound of formula I

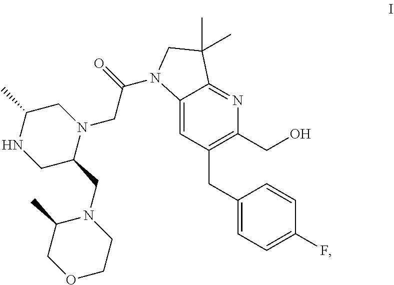 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and
said chemotherapy.

In some embodiments, provided herein is a method of treating a dexamethasone-resistant cancer (e.g., leukemia) in a subject in need thereof, the method comprising administering to the subject a compound of formula I:

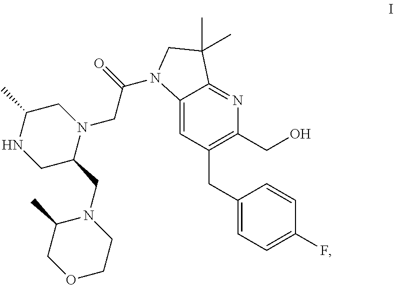 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and
dexamethasone.

In some embodiments, provided herein is a method of sensitizing a dexamethasone-resistant cancer cell line (e.g., leukemia cell line) to dexamethasone, the method comprising contacting the dexamethasone-resistant leukemia cell line with a compound of formula I:

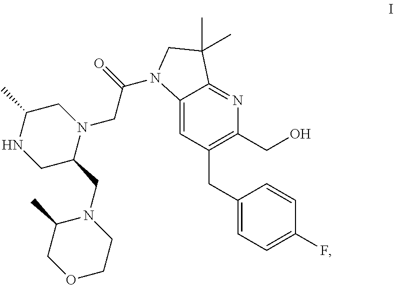 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof.

In some embodiments, provided herein is a pharmaceutical composition comprising a compound of formula I:

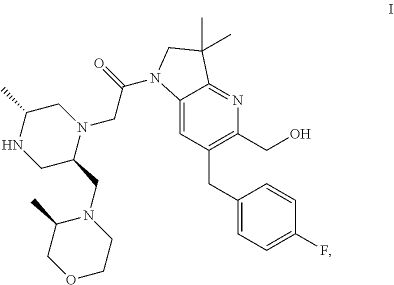 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof, and a B cell leukemia/lymphoma-2 (Bcl-2) inhibitor, or a pharmaceutically acceptable salt thereof.

In some embodiments, provided herein is a kit comprising the pharmaceutical composition.

In some embodiments, provided herein is a pharmaceutical composition for use in the treatment of cancer, the pharmaceutical composition comprising a compound of formula I:

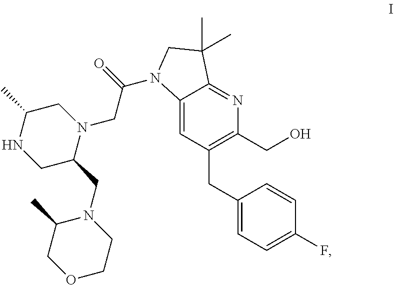 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B cell leukemia/lymphoma-2 (Bcl-2) inhibitor, or a pharmaceutically acceptable salt thereof.

In some embodiments, provided herein is a pharmaceutical composition for use in the manufacture of medicament for treating cancer, the pharmaceutical composition comprising a compound of formula I:

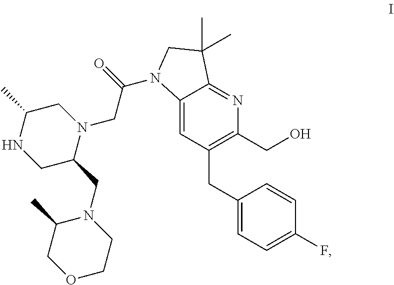 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B cell leukemia/lymphoma-2 (Bcl-2) inhibitor, or a pharmaceutically acceptable salt thereof.

In some embodiments, provided herein is a use of a pharmaceutical composition in the treatment of cancer, the pharmaceutical composition comprising a compound of formula I:

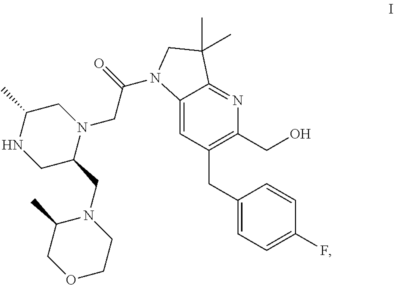 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B cell leukemia/lymphoma-2 (Bcl-2) inhibitor, or a pharmaceutically acceptable salt thereof.

In some embodiments, provided herein is a use of a pharmaceutical composition in the manufacture of a medicament for the treatment of cancer, the pharmaceutical composition comprising a compound of formula I:

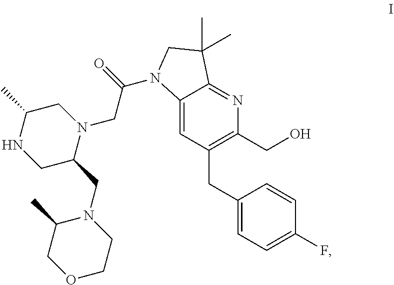 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B cell leukemia/lymphoma-2 (Bcl-2) inhibitor, or a pharmaceutically acceptable salt thereof.

### BRIEF DESCRIPTION OF DRAWINGS

FIG. 1A is a Western blot analysis showing expression of IAPs and Caspases in SUPT11 cells at the indicated time points.

FIG. 1B is a graph showing the protein levels in the Western Blot normalized toβ-Actin.

FIG. 2A is a graph showing the absolute cell count of T-ALL SUPT11 cells treated with dexamethasone (DEX) and Compound I (IAPi), each alone, and in combination, at different concentrations.

FIG. 2B is a graph showing the percent cell death of T-ALL SUPT11 cells treated with dexamethasone (DEX) and Compound I (IAPi), each alone, and in combination, at different concentrations. CI refers to the combination index.

FIG. 3A is a graph showing the absolute cell count of T-ALL CCRF-CEM cells treated with dexamethasone (DEX) and Compound I (IAPi), each alone, and in combination, at different concentrations.

FIG. 3B is a graph showing the percent cell death of T-ALL CCRF-CEM cells treated with dexamethasone (DEX) and Compound I (IAPi), each alone, and in combination, at different concentrations. CI refers to the combination index.

FIG. 4A is a graph showing the percent cell death of PDX-derived cells (CD45⁺CD7⁺CD19⁻) treated with dexamethasone (DEX) and Compound I (IAPi), each alone, and in combination, at different concentrations.

FIG. 4B is a graph showing the percent cell death of leukemia initiating cells (LICs, CD45⁺CD7⁺CD19⁻CD34⁺) treated with dexamethasone (DEX) and Compound I (IAPi), each alone, and in combination, at different concentrations.

FIG. 5A is a graph showing the absolute cell count of T-ALL LOUCY cells treated with Compound II (ABT199) and Compound I (IAPi), each alone, and in combination, at different concentrations.

FIG. 5B is a graph showing the absolute cell count of T-ALL LOUCY cells treated with Compound II (ABT199) and Compound I (IAPi), each alone, and in combination, at different concentrations. CI refers to the combination index.

FIG. 6A is a graph showing the percent cell death of PDX-derived cells (CD45⁺CD7⁺CD19⁻) treated with Compound II (ABT199) and Compound I (IAPi), each alone, and in combination, at different concentrations.

FIG. 6B is a graph showing the percent cell death of leukemia initiating cells (LICs, CD45⁺CD7⁺CD19⁻CD34⁺) treated with Compound II (ABT199) and Compound I (IAPi), each alone, and in combination, at different concentrations.

FIG. 7 is a Western blot analysis showing decreased expression of cIAP2 and Poly (ADP-ribose) polymerase (PARP) and increased cleaved caspase-7 in LOUCY cells in response to Compound I (IAPi) alone and in combination with Compound II (ABT199).

FIG. 8A is a graph showing the absolute cell count of T-ALL CCRF-CEM cells treated with Compound II (ABT199), Compound I (IAPi), and dexamethasone (DEX) each alone, and in triple combination, at different concentrations.

FIG. 8B is a graph showing the percentage cell death of T-ALL CCRF-CEM cells treated with Compound II (ABT199), Compound I (IAPi), and dexamethasone (DEX) each alone, and in triple combination, at different concentrations. CI refers to the combination index.

FIG. 9A is a graph showing the absolute cell count of T-ALL SUPT11 cells treated with Compound II (ABT199), Compound I (IAPi), and dexamethasone (DEX) each alone, and in triple combination, at different concentrations.

FIG. 9B is a graph showing the percentage cell death of T-ALL SUPT11 cells treated with Compound II (ABT199), Compound I (IAPi), and dexamethasone (DEX) each alone, and in triple combination, at different concentrations. CI refers to the combination index.

FIG. 10A is a graph showing the absolute cell count in a primary T-ALL patient sample treated with Compound II (ABT199), Compound I (IAPi), and dexamethasone (DEX) each alone, and in triple combination, at different concentrations.

FIG. 10B is a graph showing the percentage cell death in a primary T-ALL patient sample treated with Compound II (ABT199), Compound I (IAPi), and dexamethasone (DEX) each alone, and in triple combination, at different concentrations. Cell proliferation and apoptosis was measured in stem/progenitor (CD34+ve) cells by flow-cytometry based bead count and AnnexinV binding assay.

FIG. 11A is a single cell proteomics analysis showing the levels of Ki-67, cleaved PARP and cleaved caspase 3 in SUPT11 cells, 48 hours after the treatment with dexamethasone (DEX) and Compound I (IAPi), each alone, and in combination.

FIG. 11B is a single cell proteomics analysis showing multiple surface and intracellular molecules involved in apoptosis, proliferation and stress response, 48 hours after the treatment with dexamethasone (DEX) and Compound I (IAPi), each alone, and in combination.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, lactic acid (i.e. L-(+)-lactic acid, D-(−)-lactic acid, DL-lactic acid), propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri (iso-propyl) amine, tri (n-propyl) amine, ethanolamine, 2-dimethylaminoethanol, piperazine, piperidine, morpholine, N-ethylpiperidine, and the like.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Alkenyl” refers to an alkyl group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

“Haloalkyl” refers to an alkyl group as defined above and “haloalkoxy” refers to an alkoxy group as defined above, wherein one or more hydrogen atoms of the alkyl or alkoxy group are replaced by a halogen.

As used herein, the term “amino” refers to amine of formula —N(R^{N})₂, where each RN is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, —NH₂, —NH(alkyl), —N(alkyl)₂, alkyl, alkenyl, alkynyl, haloalkyl, alkoxy, or haloalkoxy).

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C3-12 cycloalkyl), 3 to 10 ring carbon atoms (i.e., C₃₋₁₀ cycloalkyl), 3 to 8 ring carbon atoms (i.e., C3-8 cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Halogen” or “halo” includes fluoro, chloro, bromo, and iodo.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Examples of the fused-heteroaryl rings include, but are not limited to, benzo [d]thiazolyl, quinolinyl, isoquinolinyl, benzo [b]thiophenyl, indazolyl, benzo [d]imidazolyl, pyrazolo [1,5-a]pyridinyl, and imidazo [1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Hetcrocyclyl” refers to a saturated or unsaturated cyclic alkyl group, with one or more ring heteroatoms independently selected from nitrogen, oxygen and sulfur. The term “heterocyclyl” includes heterocycloalkenyl groups (i.e. the beterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include pyrrolidinyl, piperidinyl, piperazinyl, oxetanyl, dioxolanyl, azetidinyl, and morpholinyl. As used herein, the term “bridged-heterocyclyl” refers to a four-to ten-membered cyclic moiety connected at two non-adjacent atoms of the heterocyclyl with one or more (e.g. 1 or 2) four-to ten-membered cyclic moiety having at least one heteroatom where each heteroatom is independently selected from nitrogen, oxygen, and sulfur. As used herein, bridged-beterocyclyl includes bicyclic and tricyclic ring systems. Also used herein, the term “spiro-heterocyclyl” refers to a ring system in which a three-to ten-membered heterocyclyl has one or more additional ring, wherein the one or more additional ring is three-to ten-membered cycloalkyl or three-to ten-membered heterocyclyl, where a single atom of the one or more additional ring is also an atom of the three-to ten-membered heterocyclyl. Examples of the spiro-heterocyclyl rings include bicyclic and tricyclic ring systems, such as 2-oxa-7-azaspiro [3.5]nonanyl, 2-oxa-6-azaspiro [3.4]octanyl, and 6-oxa-1-azaspiro [3.3]heptanyl. Examples of the fused-heterocyclyl rings include, but are not limited to, 1,2,3,4-tetrahydroisoquinolinyl, 4,5,6,7-tetrahydrothieno [2,3-c]pyridinyl, indolinyl, and isoindolinyl, where the heterocyclyl can be bound via either ring of the fused system.

Some of the compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given hercin, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H and ¹⁴C. are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of Formula I or II in which from 1 to n hydrogens attached to a carbon atom is/are replaced by deuterium, in which n is the number of hydrogens in the molecule. Such compounds exhibit increased resistance to metabolism and are thus useful for increasing the half-life of the compound of Formula I when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An 18F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I or II.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

As used herein, “Compound I” and “compound of formula I” are used interchangeably. As used herein, “Compound II” and “compound of formula II” are used interchangeably.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

### Compounds

The compound of formula I is an IAP antagonist and is described in U.S. Pat. No. 9,783,538, which disclosure is incorporated herein by reference. Compound I is named 1-(6-(4-fluorobenzyl)-5-(hydroxymethyl)-3,3-dimethyl-2,3-dihydro-1H-pyrrolo [3,2-b]pyridin-1-yl)-2-((2R,5R)-5- methyl-2- (((R)-3-methylmorpholino)methyl)piperazin-1-yl)ethan-1-one (or alternatively 1-{6-[(4-fluorophenyl)methyl]-5-(hydroxymethyl)-3,3-dimethyl-1H,2H,3H-pyrrolo[3,2-b]pyridin-1-yl}-2-|(2R,5R)-5-methyl-2-{| (3R)-3-methylmorpholin-4-yl|methyl}piperazin-1-yl|ethan-1-one). The compound of formula I may be referred to as “Compound I” herein.

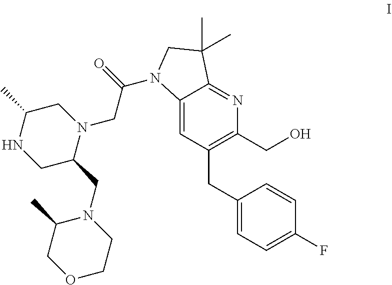 [SMILES: C[C@@H]1CN(CC(=O)N2CC(C)(C)c3nc(CO)c(Cc4ccc(F)cc4)cc32)[C@@H](CN2CCOC[C@H]2C)CN1]

The compound of formula II is a Bcl-2 inhibitor and is described in U.S. Pat. No. 8,546,399, which disclosure is incorporated herein by reference. Compound II is named venetoclax or 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl) piperazin-1-yl)-N-((3-nitro-4-(((tetrahydro-2H-pyran-4- yl)methyl)amino)phenyl)sulfonyl)benzamide (or ABT-199). The compound of formula II may be referred to as “Compound II” herein.

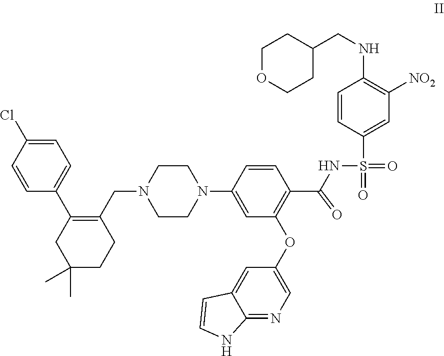 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

### Treatment Methods and Uses

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. The therapeutically effective amount may vary depending on the subject, and disease or condition being treated, the weight and age of the subject, the severity of the disease or condition, and the manner of administering, which can readily be determined by one or ordinary skill in the art.

In some embodiments, the disclosure provides a method for treating cancer in a subject comprising administering to the subject in need thereof a compound of formula I:

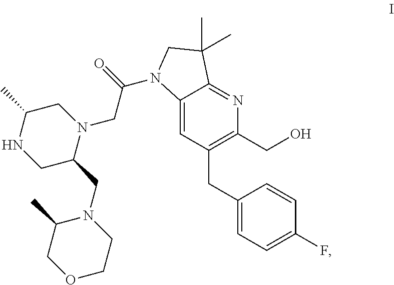 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and
a B-cell leukemia/lymphoma-2 (Bcl-2) inhibitor, or a pharmaceutically acceptable salt thereof.

In some embodiments, the Bcl-2 inhibitor is selected from venetoclax (ABT-199) and navitoclax (ABT-263). In some embodiments, the Bcl-2 inhibitor is venetoclax (ABT-199), or a pharmaceutically acceptable salt thereof. In some embodiments, the Bcl-2 inhibitor is selected from obatoclax, subatoclax, maritoclax, navitoclax, gossypol, apogossypol, ABT-737, TW-37, UMI-77, and BDA-366.

In some embodiments, provided is a method for treating cancer in a subject comprising administering to the subject in need thereof a compound of formula I:

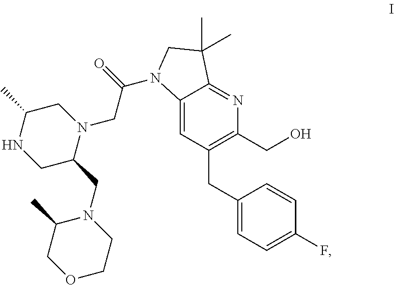 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and
a compound of formula II (venetoclax):

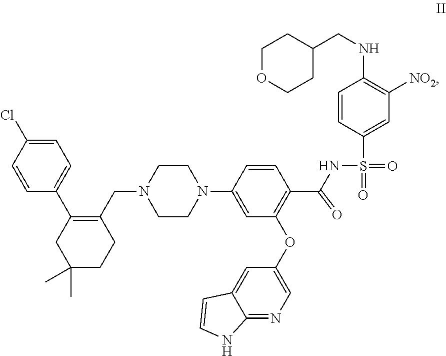 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt thereof.

In some embodiments, provided is a method for sensitizing a cancer to a chemotherapy when the cancer is refractory to said chemotherapy, the method comprising administering to a subject in need thereof a compound of formula I

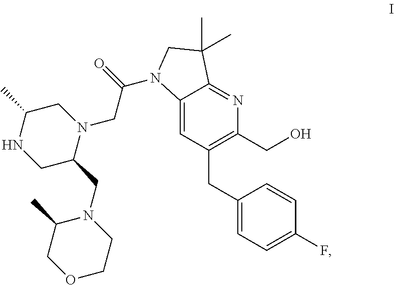 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and
said chemotherapy.

In some embodiments, the cancer is a solid tumor or a lymphoma. In some embodiments, the cancer is a hematological cancer. In some embodiments, the cancer is leukemia, lymphoma, or myeloma. In some embodiments, the cancer is myelodysplastic syndrome. In some embodiments, the cancer is non-Hodgkin's lymphoma. In some embodiments, the cancer is recurrent/metastatic head and neck squamous cell carcinoma (HNSCC), relapsed or refractory diffuse large B-cell lymphoma (DLBCL), progressive or relapsed peripheral T-cell lymphoma (PTCL), relapsed or refractory cutaneous T-cell lymphoma (CTCL), or cervical carcinoma. In some embodiments, the cancer is a leukemia. In some embodiments, the leukemia is acute myeloid (or myelogenous) leukemia (AML), chronic myeloid (or myelogenous) leukemia (CML), acute lymphocytic (or lymphoblastic) leukemia (ALL), chronic lymphocytic leukemia (CLL). In some embodiments, the leukemia is T cell acute lymphoblastic leukemia (T-ALL).

In some embodiments, the cancer is refractory to an existing chemotherapy. In some embodiments, adding an IAP antagonist (e.g., a compound of formula I) to the treatment regimen re-sensitizes the refractory cancer to the existing chemotherapy.

In some embodiments, the cancer is resistant to dexamethasone. In some embodiments, the methods further comprise administering dexamethasone to the subject. In some of such embodiments, the methods described herein sensitize a dexamethasone-resistant cancer to dexamethasone. In some of such embodiments, the dexamethasone resistant cancer is leukemia.

In some embodiments, provided is a method of treating a dexamethasone-resistant leukemia in a subject in need thereof, the method comprising administering to the subject a compound of formula I:

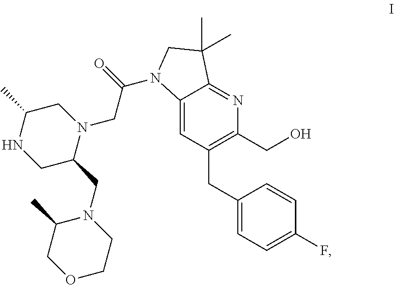 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and dexamethasone. In some embodiments, bortezomib, melphalan, prednisone may be further administered in place of or in addition to dexamethasone.

In some embodiments, provided is a method of sensitizing a dexamethasone-resistant leukemia cell line to dexamethasone, the method comprising contacting the dexamethasone-resistant leukemia cell line with a compound of formula I:

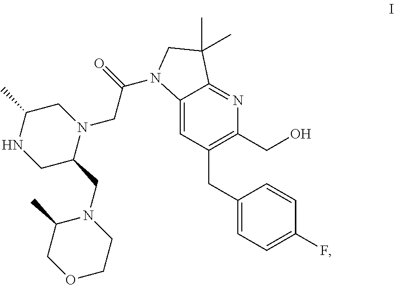 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof.

In some embodiments, provided is a method for treating T-ALL in a subject comprising administering to the subject in need thereof a compound of formula I:

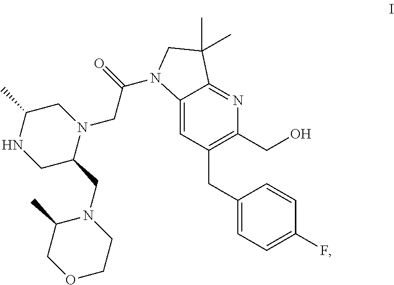 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and
a compound of formula II (venetoclax):

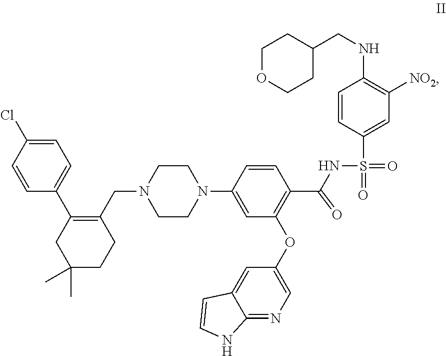 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt thereof.

In some embodiments, the method further comprises administering dexamethasone to the subject.

In some embodiments, provided is a method for treating dexamethasone-resistant T-ALL in a subject comprising administering to the subject in need thereof a compound of formula I:

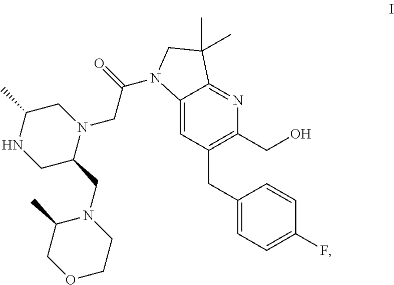 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and dexamethasone.

In some embodiments, the method further comprises administering a compound of formula II:

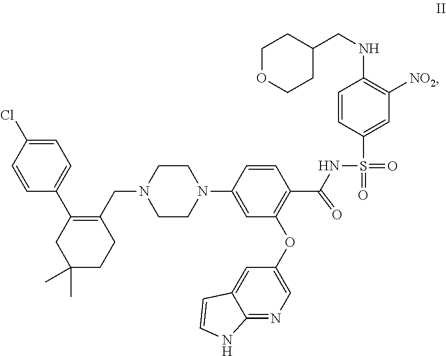 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt thereof.

In some embodiments, the compound of formula I is administered once a day for 7 consecutive days every other week of each 28-day cycle.

Provided herein is the use of a compound of formula I or a pharmaceutically acceptable salt thereof; and a B cell leukemia/lymphoma-2 (Bcl-2) inhibitor, or a pharmaceutically acceptable salt thereof, in the treatment of cancer.

Provided herein is the use of a compound of formula I or a pharmaceutically acceptable salt thereof; and a B cell leukemia/lymphoma-2 (Bcl-2) inhibitor, or a pharmaceutically acceptable salt thereof, in the treatment of cancer wherein the compound is used in combination with one or more other compounds or therapies.

Provided is a compound of formula I or a pharmaceutically acceptable salt thereof; and B cell leukemia/lymphoma-2 (Bcl-2) inhibitor, or a pharmaceutically acceptable salt thereof, for use in the treatment of cancer.

Provided is a compound of formula I or a pharmaceutically acceptable salt thereof; for use in combination with B cell leukemia/lymphoma-2 (Bcl-2) inhibitor, or a pharmaceutically acceptable salt thereof.

Provided is a compound of formula I or a pharmaceutically acceptable salt thereof; for use the treatment of cancer wherein the compound is used in combination with a B cell leukemia/lymphoma-2 (Bcl-2) inhibitor, or a pharmaceutically acceptable salt thereof.

Provided is a compound of formula I or a pharmaceutically acceptable salt thereof for use in combination therapy with a B cell leukemia/lymphoma-2 (Bcl-2) inhibitor, or a pharmaceutically acceptable salt thereof, wherein the Compound I is optionally used in combination with one or more other compounds or therapies.

Provided is the use of a compound of formula I or a pharmaceutically acceptable salt thereof, for the manufacture of a medicament for use in the treatment of cancer wherein the compound is used in combination with B cell leukemia/lymphoma-2 (Bcl-2) inhibitor, or a pharmaceutically acceptable salt thereof.

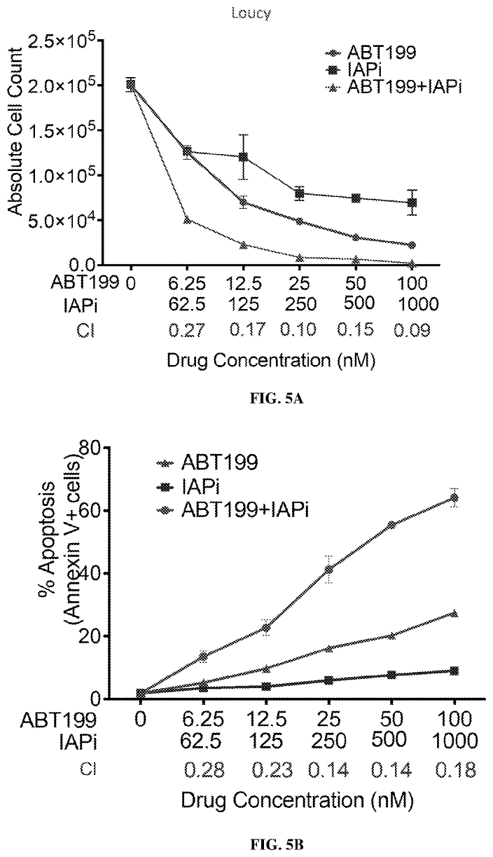

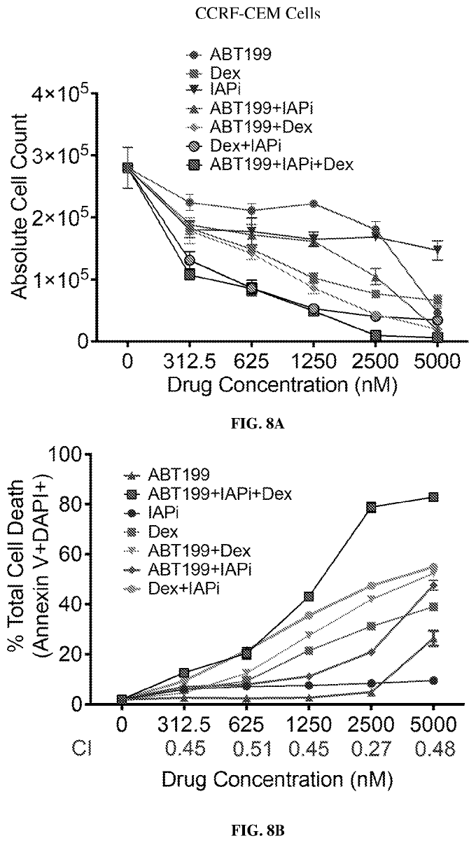

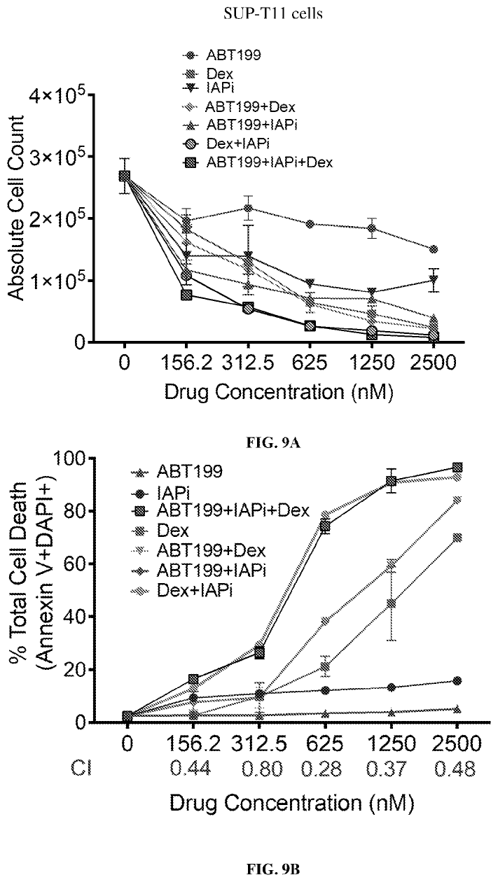

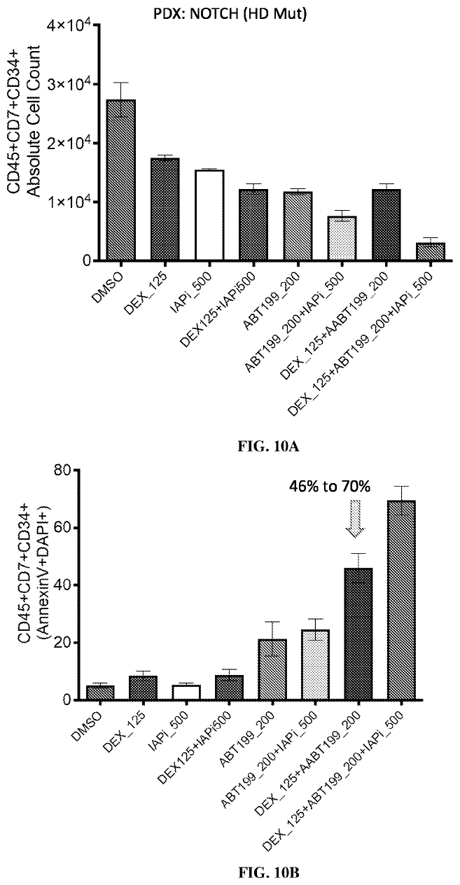

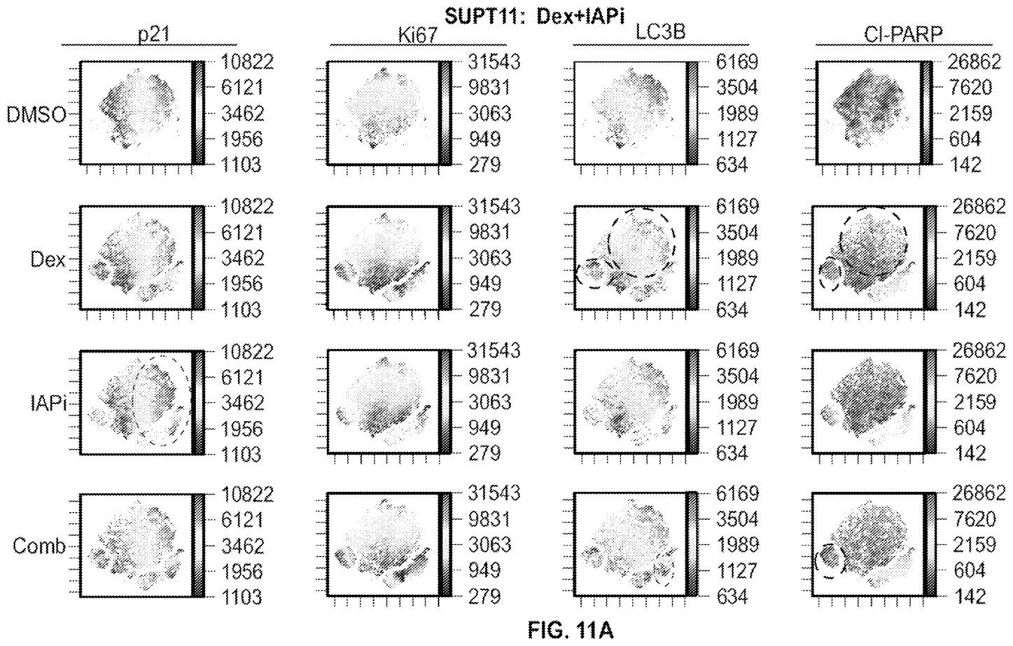

In some embodiments of the methods described herein, dexamethasone and Compound I are administered in a dose ratio of 1:1, as shown, for example, in FIG. 2A and FIG. 2B. In some embodiments of the methods described herein, Compound II and Compound I are administered in a dose ratio of 1:10 for Compound II: Compound I as shown, for example, in FIG. 5A and FIG. 5B, or, in other embodiments, in a dose ratio of 5:1, 4:1, 3:1, 2:1, 1.5:1, 1:1, 1:1.5, 1:2, 1:2.5, 1:3, 1:4, 1:5, 1:6, 1:7, 1:8, 1:9, 1:10, or 1:12.5 for Compound II: Compound I. In some embodiments of the methods described herein, Compound II, Compound I, and dexamethasone are administered in a dose ratio of 1:1:1 as shown, for example, in FIG. 8A, FIG. 8B, FIG. 9A and FIG. 9B. In some embodiments, the drug concentration of dexamethasone ranges from about 300 nM to about 5000 nM and the drug concentration of Compound I ranges from about 300 nM to about 5000 nM. In some embodiments, the drug concentration of Compound II ranges from about 6.25 nM to about 100 nM and the drug concentration of Compound I ranges from about 62.5 nM to about 1000 nM. In some embodiments, the drug concentration for each of dexamethasone, Compound I and Compound II ranges from about 300 nM to about 5000 nM.

In some embodiments of the methods described herein, navitoclax and Compound I are administered in a dose ratio of 1:10 for navitoclax: Compound I, or, in other embodiments, in a dose ratio of 5:1, 4:1, 3:1, 2:1, 1.5:1, 1:1, 1:1.5, 1:2, 1:2.5, 1:3, 1:4, 1:5, 1:6, 1:7, 1:8, 1:9, 1:10, or 1:12.5 for navitoclax: Compound I. In some embodiments of the methods described herein, navitoclax, Compound I, and dexamethasone are administered in a dose ratio of 1:1:1. In some embodiments, the drug concentration of navitoclax ranges from about 6.25 nM to about 100 nM and the drug concentration of Compound I ranges from about 62.5 nM to about 1000 nM. In some embodiments, the drug concentration for each of dexamethasone, Compound I and navitoclax ranges from about 300 nM to about 5000 nM.

In some embodiments, the method described herein may be used in combination with radiation therapy.

### Kits

Provided herein are kits that include a compound of formula I, or a pharmaceutically acceptable salt, tautomer, stereoisomer, mixture of stereoisomers, prodrug, or deuterated analog thereof, and suitable packaging. In one embodiment, the kit further includes a second therapeutic agent selected from B-cell leukemia/lymphoma-2 (Bcl-2) inhibitors described herein. In one embodiment, a kit includes a pharmaceutical composition described herein. For example, the kit may include a pharmaceutical composition comprising a compound of formula I or a pharmaceutically acceptable salt thereof, and a B-cell leukemia/lymphoma-2 (Bcl-2) inhibitors, or a pharmaceutically acceptable salt thereof. In one embodiment, a kit further includes dexamethasone in addition to the pharmaceutical composition. In one embodiment, a kit further includes instructions for use. In one aspect, a kit includes a compound of formula I, or a pharmaceutically acceptable salt, tautomer, stereoisomer, mixture of stereoisomers, prodrug, or deuterated analog thereof, and a compound of formula II, or a pharmaceutically acceptable salt, tautomer, stereoisomer, mixture of stereoisomers, prodrug, or deuterated analog thereof, and a label and/or instructions for use of the compounds in the treatment of the indications, including the diseases or conditions, described herein.

Provided herein are also articles of manufacture that include compounds or combinations of compounds described herein, or pharmaceutically acceptable salts, tautomers, stereoisomers, mixture of stercoisomers, prodrugs, or deutcrated analogs thereof in a suitable container. The container may be a vial, jar, ampoule, preloaded syringe, and intravenous bag.

### Pharmaceutical Compositions and Modes of Administration

In some embodiments, provided is a pharmaceutical composition comprising a compound of formula I:

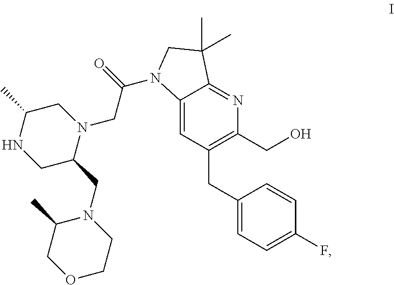 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and
a B-cell leukemia/lymphoma-2 (Bcl-2) inhibitor, or a pharmaceutically acceptable salt thereof.

In some embodiments, provided is a pharmaceutical composition comprising a compound of formula I:

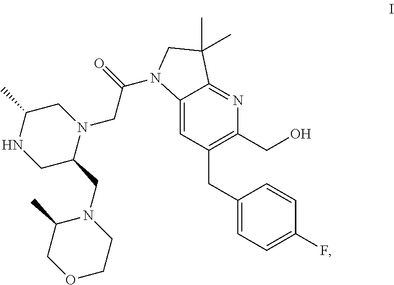 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and
a compound of formula II (venetoclax):

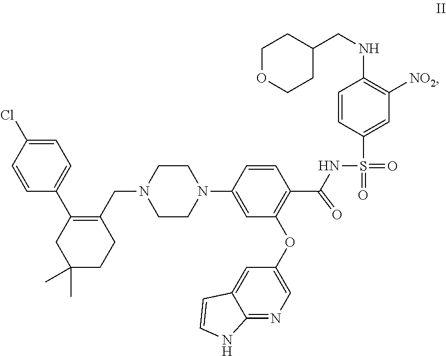 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt thereof.

Any of the pharmaceutical composition described herein may be used in the treatment of cancer and/or in the manufacture of medicament for treating cancer. In some embodiments, the cancer is a solid tumor or a lymphoma. In some embodiments, the cancer is a hematological cancer. In some embodiments, the cancer is leukemia, lymphoma, or myeloma. In some embodiments, the cancer is myelodysplastic syndrome. In some embodiments, the cancer is non-Hodgkin's lymphoma. In some embodiments, the cancer is recurrent/metastatic head and neck squamous cell carcinoma (HNSCC), relapsed or refractory diffuse large B-cell lymphoma (DLBCL), progressive or relapsed peripheral T-cell lymphoma (PTCL), relapsed or refractory cutaneous T-cell lymphoma (CTCL), or cervical carcinoma. In some embodiments, the cancer is a leukemia. In some embodiments, the leukemia is acute myeloid (or myelogenous) leukemia (AML), chronic myeloid (or myelogenous) leukemia (CML), acute lymphocytic (or lymphoblastic) leukemia (ALL), chronic lymphocytic leukemia (CLL). In some embodiments, the leukemia is T cell acute lymphoblastic leukemia (T-ALL).

In some embodiments, the cancer is refractory to an existing chemotherapy. In some embodiments, adding an IAP antagonist (e.g., a compound of formula I) to the treatment regimen re-sensitizes the refractory cancer to the existing chemotherapy.

In some embodiments, the cancer is resistant to dexamethasone. In some embodiments, the methods further comprise administering dexamethasone to the subject. In some of such embodiments, the methods described herein sensitize a dexamethasone-resistant cancer to dexamethasone. In some of such embodiments, the dexamethasone resistant cancer is leukemia.

Compounds provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. In certain embodiments, the pharmaceutical composition may be administered by intra-arterial injection, intravenously, intraperitoneally, parenterally, intramuscularly, subcutaneously, or orally.

One mode for administration is parenteral, for example, by injection or intravenously. The forms in which the pharmaceutical compositions described herein may be incorporated for administration by injection or intravenous drip include, for example, aqueous or oil suspensions, or emulsions, with sesame oil, corn oil, cottonseed oil, or peanut oil, as well as elixirs, mannitol, dextrose, or a sterile aqucous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy- benzoates; sweetening agents; and flavoring agents.

For preparing solid compositions such as tablets, the principal active ingredient may be mixed with a pharmaceutical excipient to form a solid preformulation composition containing a homogeneous mixture of a compound described herein or a pharmaceutically acceptable salt, tautomer, stereoisomer, mixture of stereoisomers, prodrug, or deuterated analog thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

### Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein (e.g. dexamethasone, Compound I, Compound II, navitoclax) per kilogram of the subject's body weight (mg/kg). Dosages of between about 0.01 and 150 mg/kg may be appropriate. In some embodiments, about 0.03 and 100 mg/kg may be appropriate. In other embodiments a dosage of between 0.1 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject. In other embodiments, a dosage may be expressed per body surface area (mg/m²).

The daily dosage may also be described as a total amount of a compound described herein (e.g. dexamethasone, Compound I, Compound II, or navitoclax) administered per dose or per day. For example, daily dosage of a compound of Formula I may be between about 1 mg and 4,000 mg, between about 2,000 to 4,000 mg/day, between about 1 to 2,000 mg/day, between about 1 to 1,000 mg/day, between about 10 to 500 mg/day, between about 10 to 300 mg/day, between about 10 to 180 mg/day, between about 20 to 500 mg/day, between about 10 to 180 mg/day, between about 50 to 300 mg/day, between about 50 to 180 mg/day, between about 75 to 200 mg/day, or between about 15 to 150 mg/day, as a free form or as a salt.

When administered orally, the total daily dosage of a compound described herein (e.g. dexamethasone, Compound I, Compound II, or navitoclax) for a human subject may be between 1 mg and 1,000 mg, between about 1,000-2,000 mg/day, between about 10-500 mg/day, between about 50-300 mg/day, between about 10-180 mg/day, between about 75-200 mg/day, or between about 100-150 mg/day.

In some embodiments, the methods comprise administering to the subject an initial daily dose of about 1 to 800 mg of a compound described herein and increasing the dose by increments until clinical efficacy is achieved. Increments of about 5, 10, 25, 50, or 100 mg can be used to increase the dose.

The compounds of the present application (e.g. dexamethasone, Compound I, Compound II, navitoclax) or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous. The compound may be administered once or more than once each day. The compound can be administered continuously (i.e. taken every day without a break for the duration of the treatment regimen). Alternatively, the compound can be administered intermittently (i.e. taken continuously for a given period such as a week, then discontinued for a period such as a week and then taken continuously for another period such as a week and so on throughout the duration of the treatment regimen). Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles. In some embodiments the first compound (e.g., Compound I) and the additional therapeutic agents (e.g., Bcl-2 inhibitor such as Compound II and/or dexamethasone) may be administered together. In some embodiments the first compound (e.g., Compound I) and the additional therapeutic agents (e.g., Bcl-2 inhibitor such as Compound II and/or dexamethasone) may be administered sequentially. In some embodiments the first compound (e.g., Compound I) and the additional therapeutic agents (e.g., Bcl-2 inhibitor such as Compound II and/or dexamethasone) may be administered using different dosing regimens. For example, dexamethasone is typically administered for 5 consecutive days in a treatment cycle, while Compound I is administered once a day for 7 consecutive days every other week of each 28-day cycle.

Ultimately, however, the quantity of compound administered and the type of composition used will be commensurate with the nature of the disease or physiological condition being treated and will be at the discretion of the physician.

### Embodiments

Embodiment 1. A method for treating cancer in a subject comprising administering to the subject in need thereof a compound of formula I:

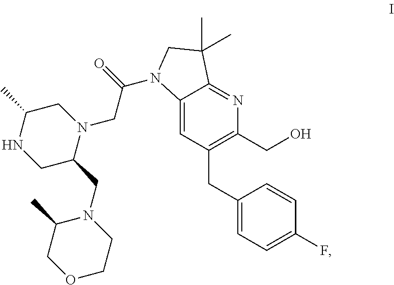 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B-cell leukemia/lymphoma-2 (Bcl-2) inhibitor, or a pharmaceutically acceptable salt thereof.

Embodiment 2. The method of embodiment 1, wherein the Bcl-2 inhibitor is venetoclax (ABT-199) or navitoclax (ABT-263).

Embodiment 3. The method of embodiment 1 or 2, wherein the cancer is a solid tumor or a lymphoma.

Embodiment 4. The method of any preceding embodiment, wherein the cancer is recurrent/metastatic head and neck squamous cell carcinoma (HNSCC), relapsed or refractory diffuse large B-cell lymphoma (DLBCL), progressive or relapsed peripheral T-cell lymphoma (PTCL), relapsed or refractory cutaneous T-cell lymphoma (CTCL), or cervical carcinoma.

Embodiment 5. The method of embodiment 1 or 2, wherein the cancer is a leukemia.

Embodiment 6. The method of embodiment 5, wherein the leukemia is T cell acute lymphoblastic leukemia (T-ALL).

Embodiment 7. The method of any preceding embodiment, wherein the cancer is resistant to dexamethasone.

Embodiment 8. The method of any preceding embodiment further comprising administering dexamethasone to the subject.

Embodiment 9. A method for treating cancer in a subject comprising administering to the subject in need thereof a compound of formula I:

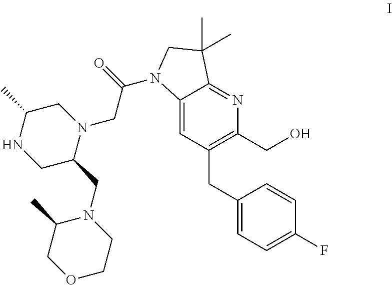 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a compound of formula II (venetoclax):

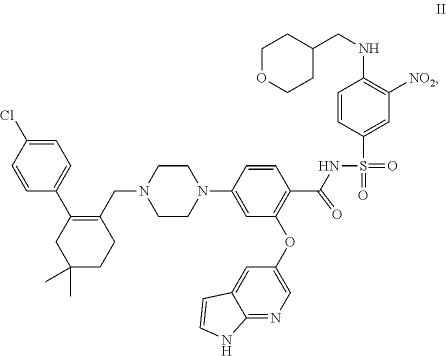 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt thereof.

Embodiment 10. The method of embodiment 9, wherein the cancer is a solid tumor or a lymphoma.

Embodiment 11. The method of embodiment 9 or 10, wherein the cancer is recurrent/metastatic head and neck squamous cell carcinoma (HNSCC), relapsed or refractory diffuse large B-cell lymphoma (DLBCL), progressive or relapsed peripheral T-cell lymphoma (PTCL), relapsed or refractory cutaneous T-cell lymphoma (CTCL), or cervical carcinoma.

Embodiment 12. The method of embodiment 9, wherein the cancer is a leukemia.

Embodiment 13. The method of embodiment 12, wherein the leukemia is T cell acute lymphoblastic leukemia (T-ALL).

Embodiment 14. The method of embodiment 13, wherein the T-ALL is resistant to dexamethasone.

Embodiment 15. The method of any one of embodiments 9-14, further comprising administering dexamethasone to the subject.

Embodiment 16. A method for sensitizing a cancer to a chemotherapy when the cancer is refractory to said chemotherapy, the method comprising administering to a subject in need thereof a compound of formula I:

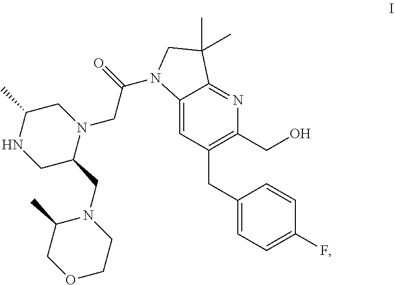 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and said chemotherapy.

Embodiment 17. A method of treating a dexamethasone-resistant leukemia in a subject in need thereof, the method comprising administering to the subject a compound of formula I:

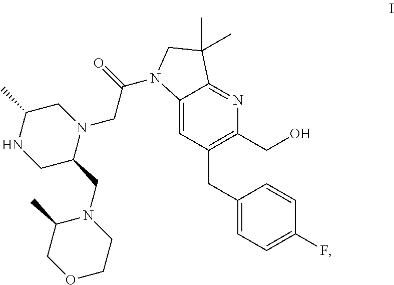 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and dexamethasone.

Embodiment 18. A method of sensitizing a dexamethasone-resistant leukemia cell line to dexamethasone, the method comprising contacting the dexamethasone-resistant leukemia cell line with a compound of formula I:

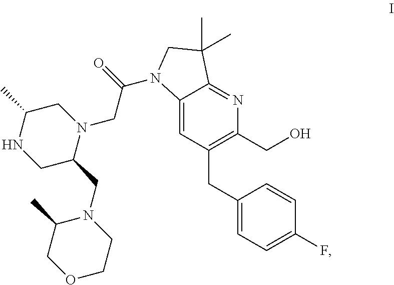 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof.

Embodiment 19. A method for treating T-ALL in a subject comprising administering to the subject in need thereof a compound of formula I:

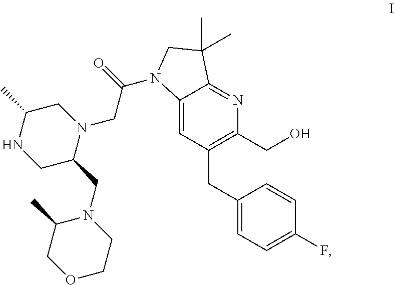 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a compound of formula II (venetoclax):

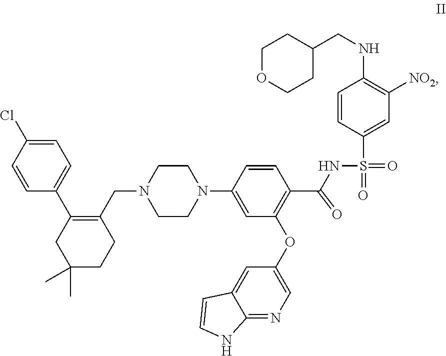 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt thereof.

Embodiment 20. The method of embodiment 19, further comprising administering dexamethasone to the subject.

Embodiment 21. A method for treating dexamethasone-resistant T-ALL in a subject comprising administering to the subject in need thereof a compound of formula I:

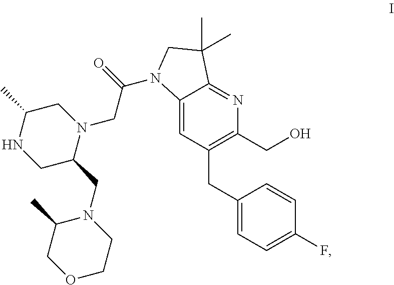 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and dexamethasone.

Embodiment 22. The method of embodiment 21, further comprising administering a compound of formula II:

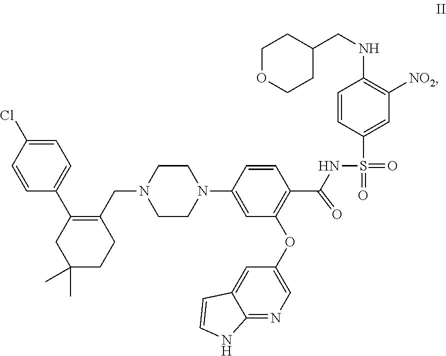 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt thereof.

Embodiment 23. The method of any preceding embodiment wherein the compound of formula I or a pharmaceutically acceptable salt thereof is administered once a day for 7 consecutive days every other week of each 28-day cycle.

Embodiment 24. The method of embodiment 23, wherein the dose of the compound of formula I or a pharmaceutically acceptable salt thereof is 10 mg to 180 mg daily.

Embodiment 25. A pharmaceutical composition comprising a compound of formula I:

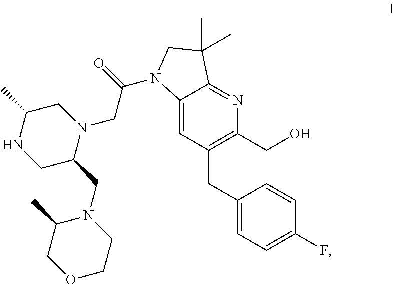 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B-cell leukemia/lymphoma-2 (Bcl-2) inhibitor, or a pharmaceutically acceptable salt thereof.

Embodiment 26. The pharmaceutical composition of embodiment 25, wherein the Bel-2 inhibitor is venetoclax (ABT-199) or navitoclax (ABT-263).

Embodiment 27. A pharmaceutical composition comprising a compound of formula I:

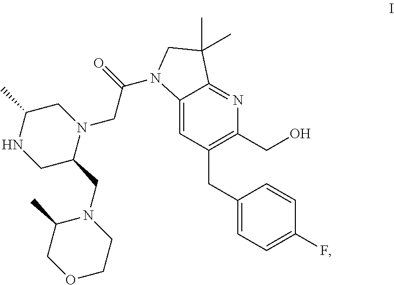 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a compound of formula II (venetoclax):

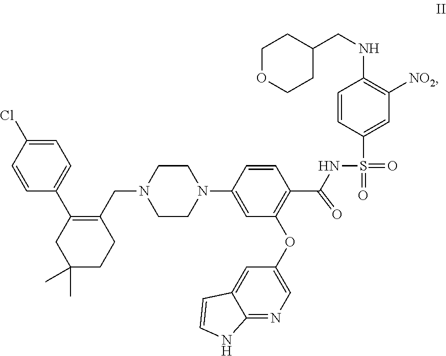 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt thereof.

Embodiment 28. The pharmaceutical composition of any one of embodiments 25 to 27, further comprising one or more pharmaceutically acceptable excipients.

Embodiment 29. A kit comprising the pharmaceutical composition of any one of embodiments 25 to 28 and dexamethasone.

Embodiment 30. A pharmaceutical composition for use in the treatment of cancer, the pharmaceutical composition comprising a compound of formula I:

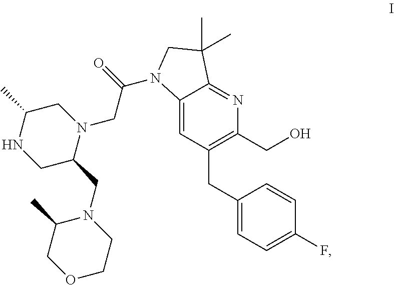 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B-cell leukemia/lymphoma-2 (Bcl-2) inhibitor, or a pharmaceutically acceptable salt thereof.

Embodiment 31. A pharmaceutical composition of a compound of formula I:

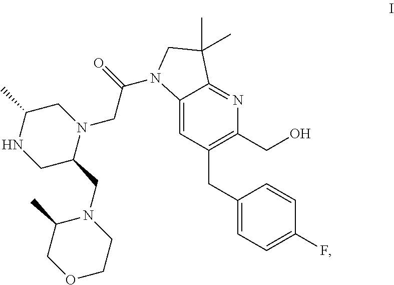 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; for use in the treatment of cancer: which is used in combination with a B-cell leukemia/lymphoma-2 (Bcl-2) inhibitor, or a pharmaceutically acceptable salt thereof.

Embodiment 32. A pharmaceutical composition for use in the manufacture of medicament for treating cancer, the pharmaceutical composition comprising a compound of formula I:

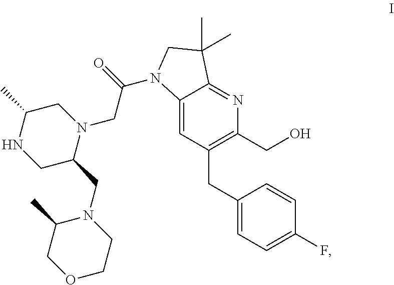 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B-cell leukemia/lymphoma-2 (Bcl-2) inhibitor, or a pharmaceutically acceptable salt thereof.

Embodiment 33. A pharmaceutical composition of a compound of formula I:

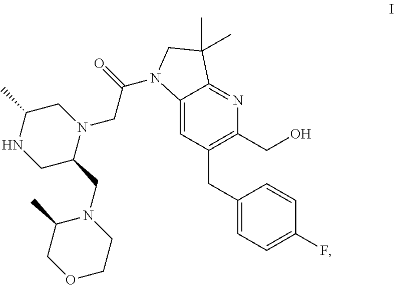 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; for use in the manufacture of medicament for treating cancer: which is used in combination with a B-cell leukemia/lymphoma-2 (Bcl-2) inhibitor, or a pharmaceutically acceptable salt thereof.

Embodiment 34. The pharmaceutical composition for use of embodiment 30 to 33, wherein the Bcl-2 inhibitor is selected from venetoclax (ABT-199) and navitoclax (ABT-263).

Embodiment 35. The pharmaceutical composition for use of any one of embodiments 30 to 34, wherein the cancer is a solid tumor or a lymphoma.

Embodiment 36. The pharmaceutical composition for use of any one of embodiments 30 to 35, wherein the cancer is recurrent/metastatic head and neck squamous cell carcinoma (HNSCC), relapsed or refractory diffuse large B-cell lymphoma (DLBCL), progressive or relapsed peripheral T-cell lymphoma (PTCL), relapsed or refractory cutaneous T-cell lymphoma (CTCL), or cervical carcinoma.

Embodiment 37. The pharmaceutical composition for use of any one of embodiments 30 to 34, wherein the cancer is a leukemia.

Embodiment 38. The pharmaceutical composition for use of embodiment 37, wherein the leukemia is T cell acute lymphoblastic leukemia (T-ALL).

Embodiment 39. The pharmaceutical composition for use of any one of embodiments 30 to 38, wherein the cancer is resistant to dexamethasone.

Embodiment 40. A use of a pharmaceutical composition in the treatment of cancer, the pharmaceutical composition comprising a compound of formula I:

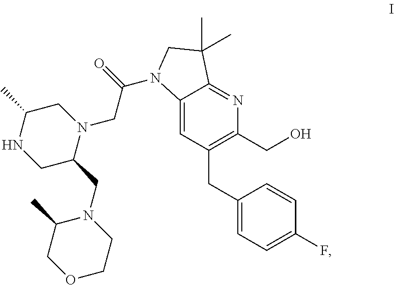 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B-cell leukemia/lymphoma-2 (Bcl-2) inhibitor, or a pharmaceutically acceptable salt thereof.

### Embodiment 41.

A use of a compound of formula I:

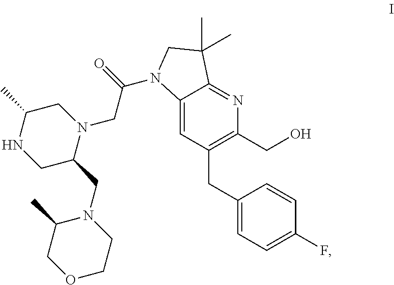 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; in the treatment of cancer: which is used in combination with a B-cell leukemia/lymphoma-2 (Bcl-2) inhibitor, or a pharmaceutically acceptable salt thereof.

Embodiment 42. A use of a pharmaceutical composition in the manufacture of a medicament for the treatment of cancer, the pharmaceutical composition comprising a compound of formula I:

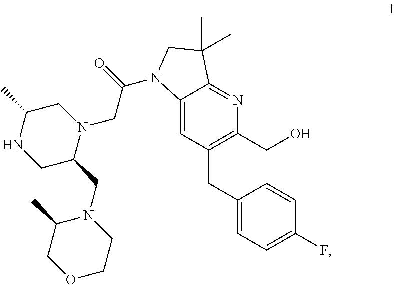 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B-cell leukemia/lymphoma-2 (Bcl-2) inhibitor, or a pharmaceutically acceptable salt thereof.

Embodiment 43. A use of a compound of formula I:

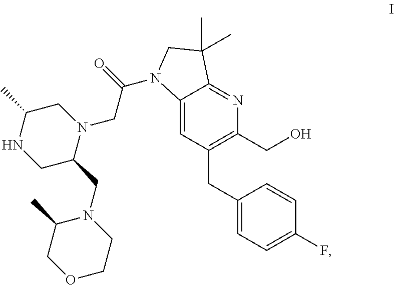 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; in the manufacture of a medicament for the treatment of cancer: which is used in combination with and a B-cell leukemia/lymphoma-2 (Bcl-2) inhibitor, or a pharmaceutically acceptable salt thereof.

Embodiment 44. The pharmaceutical composition for use of embodiments 39 to 43, wherein the Bcl-2 inhibitor is selected from venetoclax (ABT-199) and navitoclax (ABT-263).

Embodiment 45. The pharmaceutical composition for use of embodiments 39 to 43, wherein the cancer is a solid tumor or a lymphoma.

Embodiment 46. The pharmaceutical composition for use of any one of embodiments 39 to 43, wherein the cancer is recurrent/metastatic head and neck squamous cell carcinoma (HNSCC), relapsed or refractory diffuse large B-cell lymphoma (DLBCL), progressive or relapsed peripheral T-cell lymphoma (PTCL), relapsed or refractory cutaneous T-cell lymphoma (CTCL), or cervical carcinoma.

Embodiment 47. The pharmaceutical composition for use of embodiments 39 to 43, wherein the cancer is a leukemia.

Embodiment 48. The pharmaceutical composition for use of embodiment 46, wherein the leukemia is T cell acute lymphoblastic leukemia (T-ALL).

Embodiment 49. The pharmaceutical composition for use of any one of embodiments 40 to 47, wherein the cancer is resistant to dexamethasone.

Embodiment 50. A kit for use in the treatment of a cancer, the kit comprising: 1) a pharmaceutical composition comprising a compound of formula I:

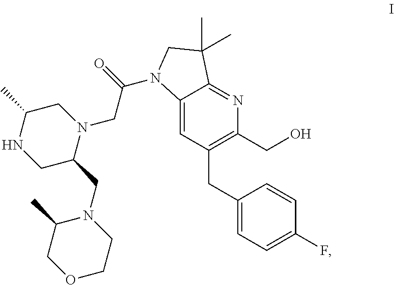 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and 2) a pharmaceutical composition comprising a B-cell leukemia/lymphoma-2 (Bcl-2) inhibitor, or a pharmaceutically acceptable salt thereof.

Embodiment 51. A medicament or an agent for use in the treatment of a cancer, the medicament or the agent comprising: a compound of formula I:

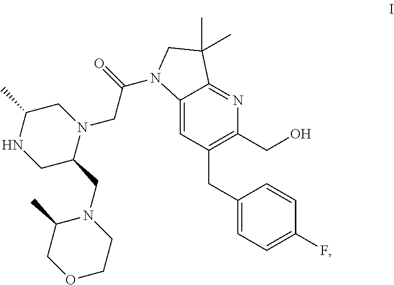 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B-cell leukemia/lymphoma-2 (Bcl-2) inhibitor, or a pharmaceutically acceptable salt thereof.

Embodiment 52. A medicament or an agent for use in the treatment of a cancer, the medicament or the agent comprising: a compound of formula I:

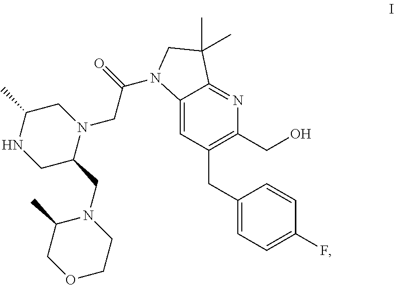 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; which is used in combination with a B-cell leukemia/lymphoma-2 (Bcl-2) inhibitor, or a pharmaceutically acceptable salt thereof.

Embodiment 53. A combination for use in the treatment of a T-cell lymphoma, the combination comprising: a compound of formula I:

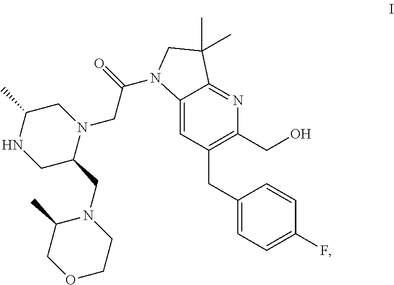 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt thereof; and a B-cell leukemia/lymphoma-2 (Bcl-2) inhibitor, or a pharmaceutically acceptable salt thereof.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

The activity of Compound I in combination with Compound II and dexamethasone (DEX) in T cell acute lymphoblastic leukemia (T-ALL) using in vitro and ex-vivo patient derived xenografts (PDX) was tested as follows.

A panel of 8 human T-ALL cell lines (SUPT11, JURKAT, CCRF-CEM, MOLT4, MOLT16, PF382, LOUCY, ALL-SIL) was used to analyze the single agent activity of Compound I. The T-All cells (0.1×10⁶/ml) were incubated with Compound I with increasing concentrations, for 5 days to determine dose response. Apoptosis was analyzed via flow cytometry (Gallios Flow Cytometer; Beckman Coulter, Fullerton, CA, USA) following staining with annexin V-APC (Biolegend, USA #640941) and DAPI (Invitrogen, Carlsbad, CA, USA). Cells were re-suspended in PBS containing CountBright beads (Invitrogen #C36950) to allow for measurement of the absolute cell number. Half-maximal inhibitory concentrations (IC50) was calculated using CalcuSyn software (BIOSOFT, Cambridge, UK). T-acute lymphoblastic leukemia cell line Loucy (LOUCY) and Stanford University Pediatric T-cell line 11 (SUPT11) were most sensitive (IC₅₀₌₁₉₀ nM and 309 nM respectively). A T lymphoblastoid cell line CCRF-CEM and a human T-ALL cell line (ALL-SIL) showed moderate sensitivity while Jurkat, MOLT16, MOLT4 and PF382 were least sensitive to Compound I.

For immunoblotting, the T-ALL cells were lysed in RIPA buffer (1% NP-40, 0.5% sodium deoxycholate, 0.1% sodium dodecyl sulfate, 50 mM Tris-Cl, pH 7.5, 150 mM NaCl) in the presence of 1X protease cocktail inhibitor. Soluble lysates were subjected to sodium dodecyl sulfate-polyacrylamide gel electrophoresis and transferred to a poly-vinylidene fluoride membrane (Bio-Rad, Hercules, CA, USA). Membranes were probed with specific antibodies. Signals were visualized using an Odyssey Infrared Imaging System (LI-COR Biosciences, Lincoln, NE, USA) and quantitated using Image Studio Lite software (LI-COR Biosciences). β-actin was used as a loading control. Immunoblotting showed decreased levels of clAP1 and clAP2 with no change in XIAP in response to Compound I as a single agent.

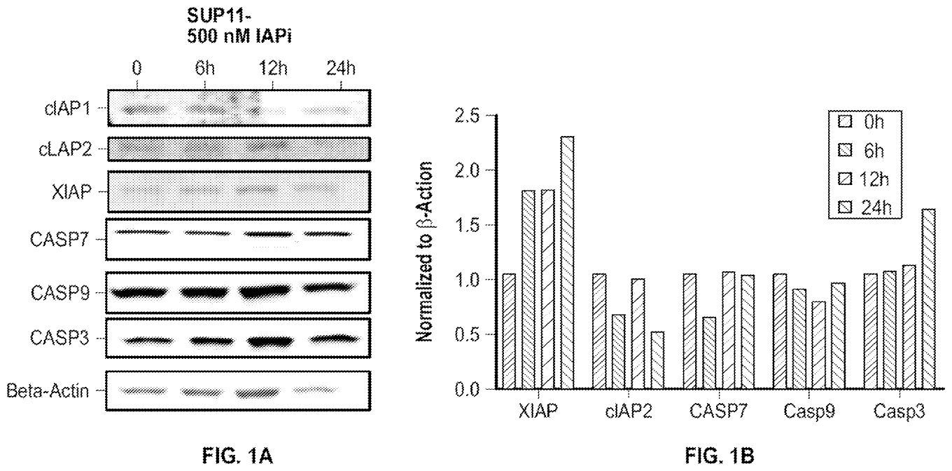

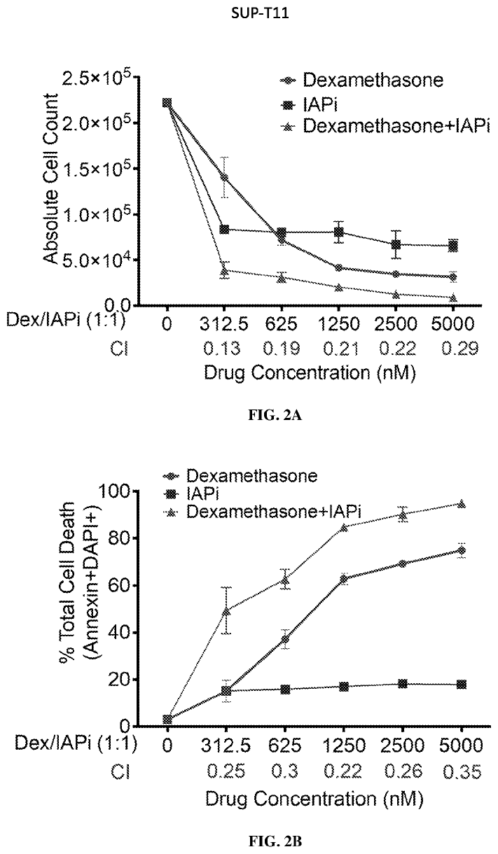

FIG. 1A shows the effect of Compound I on expression of IAPs and Caspases using Western blot analysis in SUPT11 cells at indicated time points. FIG. 1B shows the protein levels in the Western Blot normalized to β-Actin. Table 1 shows the IC₅₀ (nM) of Compound I against each of the T-ALL cell lines.

TABLE 1

| | | IC₅₀ |
|---|---|---|
| Cell Line | Mutation profile | (nM) |
| SUPT11 | NOTCH1 (wt), TP53 (pm) | 309.59 |
| JURKAT | PTEN (ins/del), TP53 (pm), | N/A |
| | CREBB(Del), NOTCH1 ins, | |
| | CDKN2A (Del), JAK1 (pm) | |
| CCRF- | KRAS, PTEN (del), TP53 (pm), | 17774 |
| CEM | FLT3(pm), FBXW7(pm), | |
| | NOTCH1 (Ins), CDKN2A (Del) | |
| MOLT4 | NOTCH1 HD (pm), PEST (del), | N/A |
| | PTEN (del), JAK1 (del/pm), | |
| | NRAS (pm), p53 (pm) | |
| MOLT16 | JAK1 (pm), PTEN ins, TP53 (pm), | N/A |
| | CDKN2A (del) | |
| PF382 | NRAS (pm), HRAS (pm), PTEN | N/A |
| | (ins), TP53 (pm), CREBB(pm), | |
| | NOTCH1 ins (pm), CDKN2A | |
| | (Del), JAK1 (pm) | |
| Loucy | JAK1 (pm), TP53 (pm), SET- | 190.1 |
| | NUP214 | |
| ALL-SIL | JAK1 (pm), (ABL1-NUP214), | 11753 |
| | NOTCH1(pm) | |

### Example 2

Next, the effect of a combination of dexamethasone (DEX) with Compound I was tested in T-ALL cell lines, using increasing concentrations of dexamethasone (DEX), Compound I, or a combination (1:1) thereof, by the method described in Example 1. The CCRF-CEM cell line is from a relapsed patient and is resistant to DEX. The combination was synergistic against the CCRF-CEM cell line, with a CI of 0.26 and cell death of 50+4% as compared to 20+3% by DEX alone, using a dose ratio of 1:1 Compound I: DEX.

Strong synergy in terms of both cytoreduction as well as apoptosis induction was observed in SUPT11 cells. Compound I sensitized SUPT11 cells to DEX with ED₅₀ value of 542 nM in combination compared to ˜2 μM with DEX treatment.

FIG. 2A shows a dose response curve for the absolute cell count for SUPT11 cells exposed to dexamethasone (DEX) and Compound I (IAPi), each alone, or in combination. FIG. 2B shows a dose response curve for the percent cell death for SUPT11 cells exposed to dexamethasone (DEX) and Compound I (IAPi), each alone, or in combination.

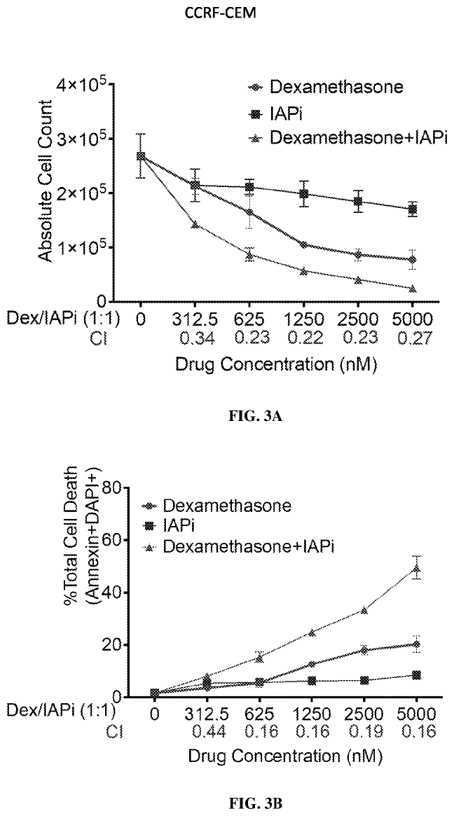

FIG. 3A shows a dosc response curve for the absolute cell count for CCRF-CEM cells exposed to dexamethasone (DEX) and Compound I (IAPi), each alone, or in combination. FIG. 3B shows a dose response curve for the percent cell death for CCRF-CEM cells exposed to dexamethasone (DEX) and Compound I (IAPi), each alone, or in combination.

As described in Example 1, absolute cell count and apoptosis was determined by flow-cytometry based bead count and AnnexinV binding assay. Combination index (CI) and IC₅₀ were determined using Calcusyn (BIOSOFT, Cambridge, UK).

Table 2 shows the IC₅₀ and ED₅₀ in SUPT11 cells.

TABLE 2

| | IC₅₀ (nM) | ED₅₀ (nM) |
|---|---|---|
| Combination | 26.7 | 542.4 |
| DEX | 350.3 | 1997.3 |
| IAPi | 11.69 | N/A |

Table 3 shows the IC₅₀ and ED₅₀ in CCRF-CEM cells

TABLE 3

| | IC₅₀ (nM) | ED₅₀ (nM) |
|---|---|---|
| Combination | 307.06 | 16046 |
| DEX | 1167.6 | 77069 |
| IAPi | 17774 | N/A |

In the MOLT16 cell line, which has a P53 point mutation and CDKN2A deletion, using a dose ratio of Compound I:DEX 1:1, the absolute cell count was decreased more in the combination as compared to either single agent IAPi or DEX, with CI of 0.05.

In PF382 cells, which have mutation in RAS, PTEN, P53, NOTCH1 and CDKN21, using a dose ratio of Compound I:DEX 1:1, though there is synergy in terms of reduction in cell number, there was no apoptosis induction using either single agents or the combination.

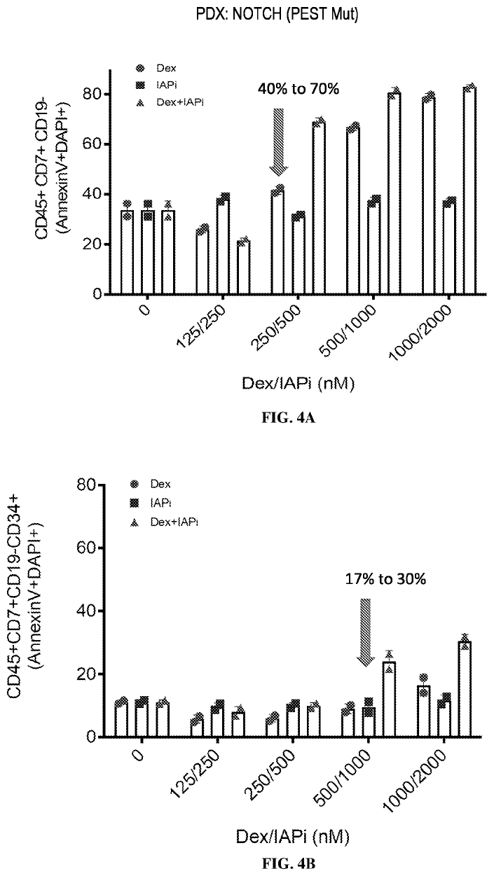

The effect of a combination of dexamethasone (DEX) with Compound I was also tested in T-ALL PDX cell lines (DFAT-72032, DFAT-28537, CBAT-37614, CBAT-93917, CBAT-44179, 6506870, D115), The T-ALL PDX cells were incubated with increasing concentrations of dexamethasone (DEX), Compound I, or a combination (1:1) thereof. After 5 days, cells were stained with annexin V-APC (Biolegend, USA #640941), CD45-PE-Cy7 (Biolegend, USA #304016), CD34-PE (BD Biosciences #348057), CD19-PER-CP (BD Biosciences #347544) and CD7-FITC (BD Biosciences (#347483) and then washed and stained with DAPI prior to analysis using flow cytometry, using increasing concentrations of dexamethasone (DEX), Compound I, or a combination (1:1) thereof. FIG. 4A shows the effect of dexamethasone (DEX) and Compound I (IAPi), each alone, and in combination, on the absolute cell count of T-ALL PDX cells. FIG. 4B shows the effect of dexamethasone (DEX) and Compound I (IAPi), each alone, and in combination, on the percent cell death of T-ALL PDX cells.

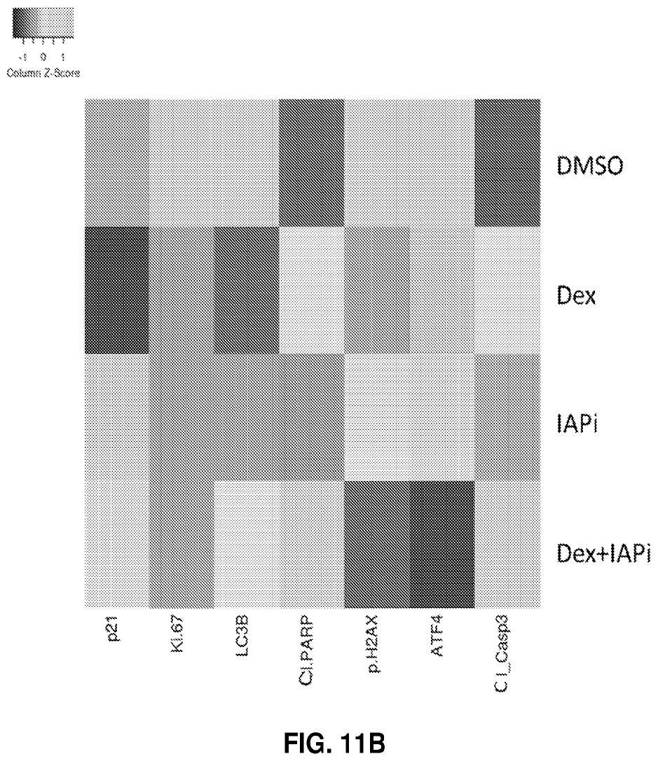

The T-ALL PDX cells were further analyzed by immunoblotting, using the method described in Example 1. FIG. 11A shows the effect of dexamethasone (DEX) and Compound I (IAPi), each alone, and in combination, on levels of cleaved PARP and cleaved caspase 3. FIG. 11B shows the effect of dexamethasone (DEX) and Compound I (IAPi), each alone, and in combination, on multiple surface and intracellular molecules involved in apoptosis, proliferation and stress response using single cell proteomics analysis at 48 h post treatment.

As shown in FIGS. 11A and 11B, Compound I (IAPi) treatment in combination with dexamethasone (DEX) increased levels of cleaved PARP and cleaved caspase 3 suggesting increased apoptosis. Simultaneous analysis of cell proliferation, stress response and DNA damage using single-cell proteomics analysis showed downregulation of proliferation (Ki-67), stress response (ATF4, LC3B) and increased levels of cleaved PARP and cleaved caspase 3, suggesting increased apoptosis from the combination of Compound I (IAPi) with DEX.

### Example 3

The efficacy of a combination of Compound I with Bcl2 inhibitor venetoclax (ABT-199,Compound II) was tested. The effect of a combination of Compound I with Bcl2 inhibitor venetoclax (ABT-199, Compound II) was tested in T-ALL cell lines, using increasing concentrations of Compound II, Compound I, or a combination (1:10) thereof, by the method described in Example 1, and the combination index (CI) was calculated. The combination of Compound I and Compound II, in a dose ratio of Compound II: Compound I of 1:10, was synergistic in the LOUCY cell line with a combination index (CI) of 0.14. Cell death was increased to 64+3% in combination as compared to 27+0.9% with Compound II alone. Western blotting, conducted using the same method described in Example 1, showed decreased cIAP2 and increased levels of cleaved caspase 7 and cleaved caspase 9 in combination as compared to Compound I alone, or Compound II alone, suggesting increased induction of apoptosis. The effect of a combination of Compound I with Bcl2 inhibitor venetoclax (ABT-199, Compound II) in T- ALL PDX cells was tested, using increasing concentrations of Compound II, Compound I, or a combination (1:10) thereof, by the method described in Example 2, The ex vivo treatment of patient derived xenograft cells (PDX-derived cells) with Compound I and Compound II was more effective than monotherapy in inducing apoptosis in CD45+bulk (46+0.7% to 63+6%, p<0.0001) and leukemia initiating cells (LICs, CD45+, CD7+, CD19, CD34+) (39+3% to 54+8%, p=0.003).

FIG. 5A shows the effect of Compound II (ABT199) and Compound I (IAPi), each alone, and in combination, on the absolute cell count of T-ALL LOUCY cells. FIG. 5B shows the effect of Compound II (ABT199) and Compound I (IAPi), each alone, and in combination, on the percentage cell death of T-ALL LOUCY cells. CI refers to the combination index.

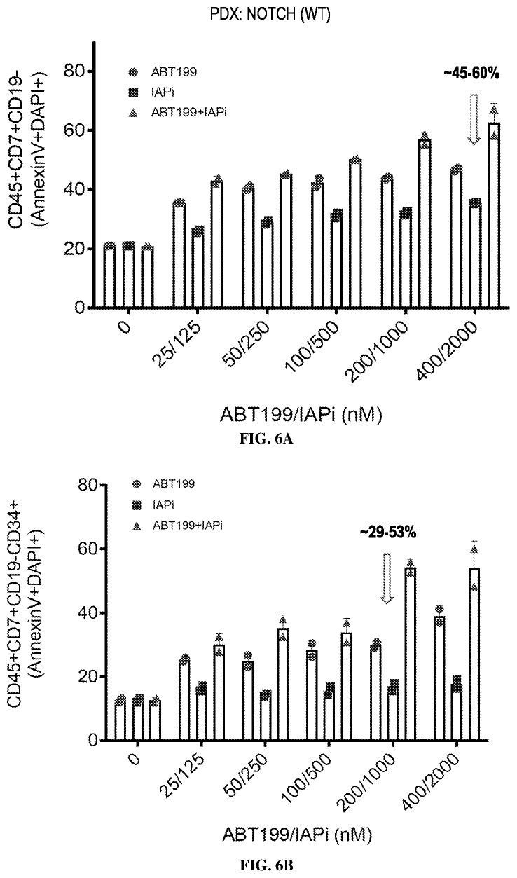

FIG. 6A shows the effect of Compound II (ABT199) and Compound I (IAPi), each alone, and in combination, on the absolute cell count of T-ALL PDX cells. FIG. 5B shows the effect of Compound II (ABT199) and Compound I (IAPi), each alone, and in combination, on the percentage cell death of T-ALL PDX cells.

As described herein, absolute cell count and apoptosis was determined by flow-cytometry based bead count and AnnexinV binding assay. Combination index and IC₅₀ were determined using Calcusyn (BIOSOFT, Cambridge, UK).

Table 4 shows the IC₅₀ and ED₅₀ in LOUCY cells.

TABLE 4

| | IC₅₀ (nM) | ED₅₀ (nM) |
|---|---|---|
| Combination | 2.27 | 48.78 |
| ABT199 | 8.18 | 314.91 |
| IAPi | 190.1 | 67838 |

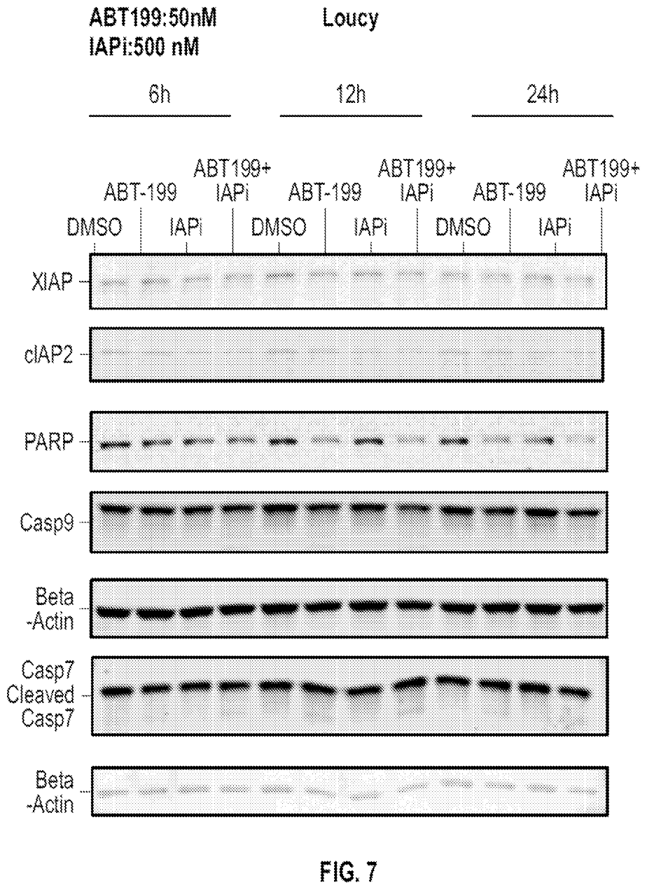

FIG. 7 shows a Western blot analysis showing decreased expression of cIAP2 and PARP and increased cleaved caspase-7 in LOUCY cells in response to Compound I (IAPi) alone and in combination with Compound II (ABT199).

In ALL-SIL cells, using a dosc ratio of 1:2.5 for Compound II: Compound I, the absolute cell count decreased more greatly with the combination compared to each individual agent alone, and the IC₅₀ values indicate synergy using the combination compared to single agents

### Example 4

The effect of a triple combination of DEX, Compound I and Compound II in a dose ratio of 1:1:1, and dual combination of DEX+Compound II on T-ALL cell lines and T-ALL PDX cells were analyzed using flow cytometry, and the combination index and IC₅₀ were determined as described in preceding examples. Immunoblotting was also conducted as described in preceding examples. A triple combination of DEX, Compound I and Compound II in a dose ratio of 1:1:1 increased apoptotic response to 82.9+1%, compared to the dual combination of DEX+Compound II where apoptosis was induced to only 52.4+2%.

Synergy in terms of both cytoreduction as well as apoptosis induction was observed in SUPT11 cells. Compound I sensitized SUPT11 cells to DEX with ED₅₀ value of 542 nM in combination as compared to ˜2 μM with only DEX treatment. Simultaneous analysis of cell proliferation, stress response and DNA damage using single-cell proteomics analysis showed downregulation of proliferation (Ki-67), stress response (ATF4, LC3B) and increased levels of cleaved PARP, cleaved caspase 3 suggesting increased apoptosis in response to the combination of Compound I with DEX. Ex vivo treatment of PDX with Compound I enhanced the cytotoxic effect of DEX in CD45+bulk (58±2% to 71±0.1%, p<0.0001) and LICs (62±2% to 78±0.9%, p<0.0001).

FIG. 8A shows the effect of Compound II (ABT199), Compound I (IAPi), and dexamethasone (DEX) each alone, and in triple combination, on the absolute cell count of T-ALL CCRF-CEM cells. FIG. 8B shows the effect of Compound II (ABT199), Compound I (IAPi), and dexamethasone (DEX) each alone, and in triple combination, on the percentage cell death of T-ALL CCRF-CEM cells. CI refers to the combination index.

FIG. 9A shows the effect of Compound II (ABT199), Compound I (IAPi), and dexamethasone (DEX) each alone, and in triple combination, on the absolute cell count of T-ALL SUPT11 cells. FIG. 9B shows the effect of Compound II (ABT199), Compound I (IAPi), and dexamethasone (DEX) each alone, and in triple combination, on the percentage cell death of T-ALL SUPT11 cells. CI refers to the combination index.

As described herein, absolute cell count and apoptosis was determined by flow-cytometry based bead count and AnnexinV binding assay. Combination index was determined using Calcusyn (BIOSOFT, Cambridge, UK). The combination index is for the combination of Compound I (IAPi) to the dual combination of Compound II (ABT199) and dexamethasone (ABT199+DEX).

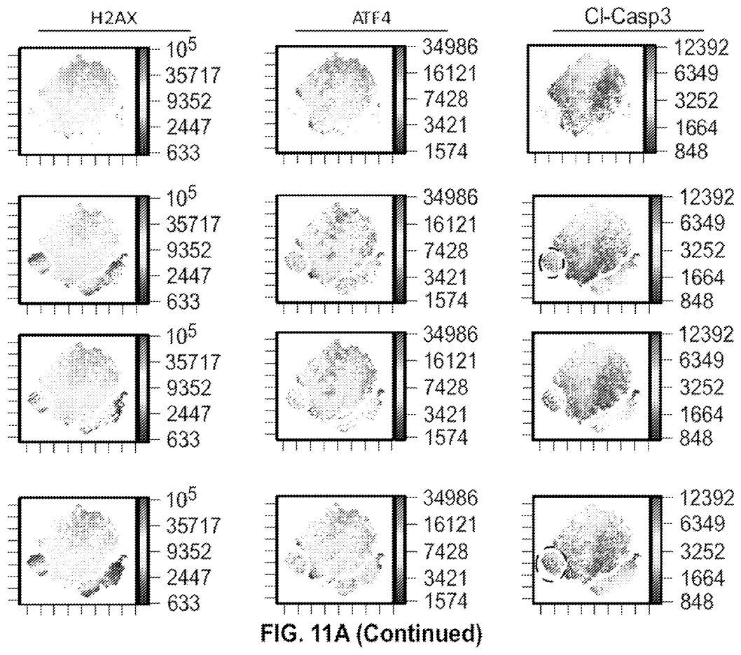

FIG. 10A shows the effect of Compound II (ABT199), Compound I (IAPi), and dexamethasone (DEX) each alone, and in triple combination, on absolute cell count in a primary T-ALL patient sample. FIG. 10B shows the effect of Compound II (ABT199), Compound I (IAPi), and dexamethasone (DEX) each alone, and in triple combination, on the percentage cell death in a primary T-ALL patient sample.

In terms of apoptosis induction, the triple combination of Compound I, Compound II, and DEX, was effective. The combination index for the triple combination was less than 0.5 suggesting good synergy. A dual combination of DEX+IAPi (Compound I) was also effective. A dual combination of DEX+ABT199 (Compound II) (data not shown) was moderately effective.

It was confirmed that Compound I synergizes with the anti-leukemic activity of Compound II and DEX, establishing a therapeutic rationale for IAP antagonists in treatment of cancers. The data shows a decrease in cIAP-2 and increase in cleaved Caspase-7 in response to single agent Compound I, and in combination with ABT199. Further, the triple combination of Compound I, ABT199 and DEX induced much greater degree of apoptosis in leukemia initiating cells (LICs, CD45⁺, CD7⁺, CD19⁻, CD34⁺), as compared to mono-and/or dual-therapy, as shown in FIGS. 10A-10B. It is contemplated that IAP inhibition (e.g., with Compound I) in combination with Bcl2 inhibition (e.g. Compound II) and dexamethasone shifts the cytostatic effect of single agents to cytotoxic effect in T-ALL progenitors and increases apoptosis in LICs.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for treating cancer in a subject comprising administering to the subject in need thereof a compound of formula I: 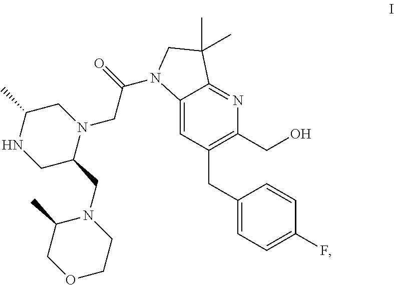 [SMILES: C[C@@H]1CN(CC(=O)N2CC(C)(C)c3nc(CO)c(Cc4ccc(F)cc4)cc32)[C@@H](CN2CCOC[C@H]2C)CN1]
or a pharmaceutically acceptable salt thereof; and
a B-cell leukemia/lymphoma-2 (Bcl-2) inhibitor, or a pharmaceutically acceptable salt thereof.

2. The method of claim 1, wherein the Bcl-2 inhibitor is venetoclax (ABT-199) or navitoclax (ABT-263) or a pharmaceutically acceptable salt thereof.

3. The method of claim 1, wherein the cancer is a solid tumor or a lymphoma.

4. The method of claim 1, wherein the cancer is recurrent/metastatic head and neck squamous cell carcinoma (HNSCC), relapsed or refractory diffuse large B-cell lymphoma (DLBCL), progressive or relapsed peripheral T-cell lymphoma (PTCL), relapsed or refractory cutaneous T-cell lymphoma (CTCL), or cervical carcinoma.

5. The method of claim 1, wherein the cancer is a leukemia.

6. The method of claim 5, wherein the leukemia is T cell acute lymphoblastic leukemia (T-ALL).

7. The method of claim 1, wherein the cancer is resistant to dexamethasone.

8. The method of claim 1, further comprising administering dexamethasone to the subject.

9. A method for sensitizing a cancer to a chemotherapy when the cancer is refractory to said chemotherapy, the method comprising administering to a subject in need thereof a compound of formula I 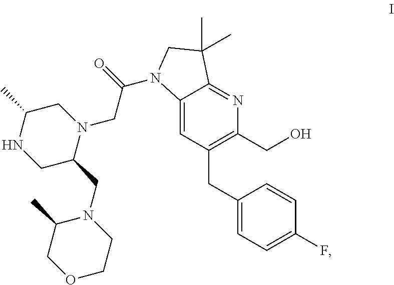 [SMILES: C[C@@H]1CN(CC(=O)N2CC(C)(C)c3nc(CO)c(Cc4ccc(F)cc4)cc32)[C@@H](CN2CCOC[C@H]2C)CN1]
or a pharmaceutically acceptable salt thereof; and
said chemotherapy.

10. The method of claim 9, wherein the chemotherapy is dexamethasone administration, and the cancer is a dexamethasone-resistant leukemia.

11. A method of sensitizing a dexamethasone-resistant leukemia cell line to dexamethasone, the method comprising contacting the dexamethasone-resistant leukemia cell line with a compound of formula I: 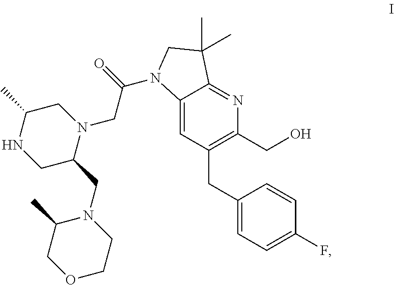 [SMILES: C[C@@H]1CN(CC(=O)N2CC(C)(C)c3nc(CO)c(Cc4ccc(F)cc4)cc32)[C@@H](CN2CCOC[C@H]2C)CN1]
or a pharmaceutically acceptable salt thereof.

12. The method of claim 6,
or a pharmaceutically acceptable salt thereof; and
wherein the B-cell leukemia/lymphoma-2 (Bcl-2) inhibitor is a compound of formula II (venetoclax): 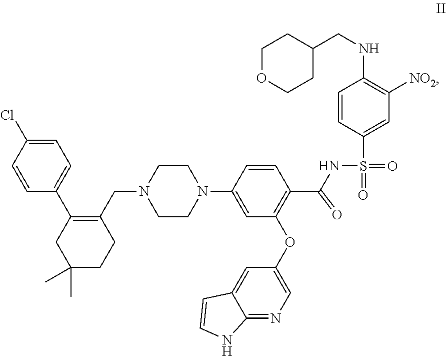 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]
or a pharmaceutically acceptable salt thereof.

13. The method of claim 12, further comprising administering dexamethasone to the subject.

14. The method of claim 10, wherein the dexamethasone-resistant leukemia is dexamethasone-resistant T-ALL.

15. The method of claim 14, further comprising administering a compound of formula II: 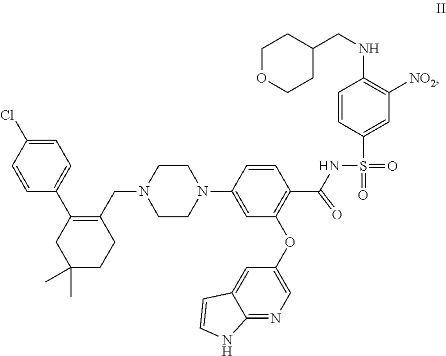 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]
or a pharmaceutically acceptable salt thereof.

16. The method of claim 1, wherein the compound of formula I or a pharmaceutically acceptable salt thereof is administered once a day for 7 consecutive days every other week of each 28-day cycle.

17-31. (canceled)

32. The method of claim 1, wherein the compound is administered sequentially or together with a B-cell leukemia/lymphoma-2 (Bcl-2) inhibitor a pharmaceutically acceptable salt thereof.

33. The method of claim 9, wherein the compound is administered sequentially or together with said chemotherapy.

34. A kit comprising a compound of formula I 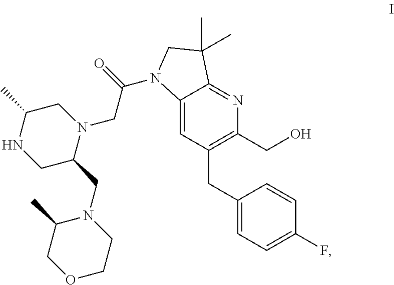 [SMILES: C[C@@H]1CN(CC(=O)N2CC(C)(C)c3nc(CO)c(Cc4ccc(F)cc4)cc32)[C@@H](CN2CCOC[C@H]2C)CN1]
or a pharmaceutically acceptable salt thereof, and a B-cell leukemia/lymphoma-2 (Bcl-2) inhibitor a pharmaceutically acceptable salt thereof.

35. The kit according to claim 34, wherein the kit further comprises a chemotherapy.
